(19) United States
(12) Patent Application Publication
(10) Pub. No.: US 2009/0023985 A1
(43) Pub. Date: Jan. 22, 2009

(54) ENDOLUMINAL INSTRUMENT MANAGEMENT SYSTEM

(75) Inventor: Richard C. Ewers, Fullerton, CA (US)

Correspondence Address: LEVINE BAGADE HAN LLP, 2483 EAST BAYSHORE ROAD, SUITE 100, PALO ALTO, CA, 94303, US

(73) Assignee: USGI MEDICAL, INC., San Clemente, CA (US)

(21) Appl. No.: 12/138,348

(22) Filed: Jun. 12, 2008

Related U.S. Application Data
(60) Provisional application No. 60/944,073, filed on Jun. 14, 2007.

(51) Int. Cl. A61B 1/00

(52) U.S. Cl. 600/104

(57) ABSTRACT

Endoluminal instrument management systems are described herein which allow one or more operators to manage multiple different instruments utilized in endoluminal procedures. Responsibility for instrumentation management between one or more operators may be configured such that a first set of instruments is controlled by a primary operator and a second set of instruments is controlled by a secondary operator. The division of instrumentation may be facilitated by the use of separated instrumentation platforms or a single platform which separates each instrument for use by the primary operator. Such platforms may be configured as trays, instrument support arms, multi-instrument channels, as well as rigidized portions of instruments to facilitate its handling, among others.

### CROSS-REFERENCE TO RELATED APPLICATION

This application claims priority to and the benefit of the filing date of U.S. Provisional Patent Application Ser. No. 60/944,073 (Attorney Docket No. USGIPZ05600), filed Jun. 14, 2007. The foregoing application is hereby incorporated by reference in its entirety.

### FIELD OF THE INVENTION

The present invention relates to methods and apparatus for managing multiple instruments and tools used during endoluminal procedures. More particularly, the present invention relates to methods and devices used to facilitate multiple instrument management and their use during procedures where these instruments are advanced endoluminally into a patient body via one or more orifices.

### BACKGROUND OF THE INVENTION

Endoluminal procedures and surgery typically entail the advancement and use of one or more instruments through the natural orifices of a patient body and through the tortuous endoluminal pathways to reach the tissue regions of interest. Even procedures performed in body spaces within the patient may entail entry and advancement through one or more openings created in the patient body to gain entry into the desired body space, e.g., entry through a percutaneous opening or a gastrotomy to gain entry into the peritoneal space of the patient.

Because endoluminal surgery may involve the use of multiple instruments through a single conduit into the patient body, efficient management and use of these instruments may be difficult in part not only because of the number of instruments utilized, but also because these multiple instruments typically converge from a single conduit, which may be limited by the cross-sectional profile of the body lumen, organ, orifice, passageway, etc., in which the conduit is disposed. At the same time, advances in therapeutic endoscopy have led to an increase in the complexity of endoluminal operations attempted, as well as the complexity of tools advanced through the working lumens of these conduits.

Because of the number of instruments which converge typically from a single conduit, difficulties may arise in effectively handling and managing the placement, positioning, and use of these multiple instruments in an effective and safe manner.

For example, flexible endoscopes and flexible endoscopic instruments provide the ability for an operator to intubate the patient and to provide therapy to the internal anatomy by way of non-straight access pathways. Typical endoscopes have the ability to steer at the tip and provide light and visualization, gas insufflation, and lens rinsing. Such endoscopes will typically include one or two instrument channels. These instrument channels include an angled interface on the handle of the endoscope having a bend of about 45 degrees on a relatively short section of the handle. One result of this configuration is that any instrument that is to be inserted into the endoscope instrument channel must include a shaft that is flexible over its entire length.

Accordingly, there is a need for methods and devices for facilitating the introduction and management of all the instruments advanced through the relatively small conduits for performing endoluminal procedures.

### SUMMARY OF THE INVENTION

An endoluminal tissue manipulation assembly may comprise, at least in part, a distal end effector assembly disposed or positionable at a distal end of a flexible and elongate body. Examples are described in further detail in U.S. Pat. Pub. No. 2005/0272977 A1, which is incorporated herein by reference in its entirety. A handle assembly may be connected to a proximal end of the elongate body and include a number of features or controls for articulating and/or manipulating both the elongate body and/or the distal end effector assembly. The elongate body may optionally utilize a plurality of locking or lockable links nested in series along the length of the elongate body which enable the elongate body to transition between a flexible state and a rigidized or shape-locked configuration. Details of such a shape-lockable body may be seen in further detail in U.S. Pat. Nos. 6,783,491; 6,790,173; and 6,837,847, each of which is incorporated herein by reference in its entirety.

One or more various instruments may be passed through the elongate body for deployment through its distal end by introducing the instruments through one or more corresponding tool ports located in the handle assembly. One instrument in particular which may be used to endoluminally visualize procedures and tissue regions of interest may include an endoscope or imaging system having a flexible shaft which may be introduced into the elongate body via a side port, e.g., Y-Port, located along the elongate body and distal to the handle assembly.

Because of the number of different instruments and the different types of tools which may be utilized in the endoluminal tissue manipulation assembly, tool or instrumentation management is one consideration for the practitioner or practitioners to facilitate efficient surgical and/or endoscopic procedures when performed upon a patient. Additionally, the division of responsibility for instrumentation management between one or more practitioners is highly desirable to ensure patient safety and procedure facilitation.

One configuration for device management is to divide the operation and articulation of instruments between a primary operator and at least a secondary operator. A first set of instruments, including operation of the elongate body and transitioning the elongate body between its rigid and flexible states may be controlled via a primary operator while a second set of instruments, including operation and control of the endoscope or imaging system through the Y-Port and operation of the one or more tools may be controlled via a secondary operator who may be positioned along side or proximate to the primary operator. Accordingly, the second set of instruments may be positioned away from the first set of instruments to facilitate procedures.

Such a configuration may include the use of a control bundle or umbilicus connected to the handle or to the elongate body via an umbilicus port. The control umbilicus may be a bundle of individual lumens or a flexible tubular member having individual lumens routed therethrough which connect to corresponding lumens routed through the elongate body. Use of a single umbilicus extending from the elongate body or handle may facilitate the handling and positioning of multiple instruments for a secondary operator.

Yet another configuration for instrument positioning and management is use of an articulatable tray for use by the primary or secondary operator or another person. The tray may be a stand-alone tray or one attached to a patient bed or operating table via an articulatable support arm and may have one or more holders thereon to temporarily hold onto or retain a corresponding tool. The support arm may be pivoted or translated relative to the table in any number of positions to facilitate use as a support for the second set of instruments.

Alternatively, a single support arm may be utilized to manipulate and manage the entire system. The support arm may be attached to the table with a pivotable or static connection or it may be a stand-alone member. The support arm may further have one or more instrument booms which project or extend over the handle assembly where each of the instrument booms may be attachable either directly or via extendable and/or retractable instrument supports to a corresponding instrument extending from the elongate body or handle.

Another variation may include a curved or arched support platform attachable directly to a table via one or more pivotable joints. The platform may allow for angular adjustment between a horizontal and a vertical position and may also be translatably connected to the table via a slidable connection to allow for sliding adjustment as well as to allow for height adjustment relative to the patient body. Each of the instruments may be temporarily attached or connected via one or more corresponding attachments positioned along the platform.

Another platform to facilitate use of the system with a patient is an angled or curved support arm having a restraining strap which may be gently secured to a patient's head to maintain the head in a stable manner, particularly when the elongate body has been inserted through the patient's mouth.

Another variation may include a holder interface that is used in combination with a support platform or support arm to hold or support the endoluminal tissue manipulation assembly in a manner that facilitates rotational or translational movement of the assembly. In one embodiment, the holder interface includes a rotating clamp that is attached to the endoscope or endoluminal access device. The rotating clamp may include a post or other connector suitable for connecting the clamp to the support platform or support arm. The rotating clamp provides the operator with the ability to rotate the endoscope or endoluminal access device relative to the support arm. The rotating clamp may also include on/off or variable resistance rotation locks. In other embodiments, the holder interface includes a linear travel member that provides controlled input or side-to-side movement of the endoscope or endoluminal access device relative to the support arm. Still other embodiments include combinations of linear and rotational movement.

Aside from table-mounted or stand-alone instrument supporting members, additional instrument management systems may be employed which a single operator or user may utilize. A multi-instrument support arm extending proximally from the handle assembly may generally comprise a stiffened multi-lumen channel having a straight support channel extending proximally and one or more angled or curved support channels projecting at an angle therefrom support arm. Because the multi-instrument support arm is relatively stiff, it may be engaged to the handle assembly and used to support and separate its respective instruments leaving the operator to hold a single handle during a procedure. Other variations may include a pivoting multi-instrument support having one or more individual instrument ports pivotably positioned within an open channel. Still other variations may include a manifold that is attachable to the handle assembly and that supports one or more elongated straight docking sections each defining a substantially straight lumen for receiving an instrument shaft in a slidable docking configuration.

Yet another instrument management system may include a detachable instrument clip attached to a distal portion of the handle. The instrument clip may be opened and closed to securely receive the proximal portions of the one or more flexible shafts inserted through the tool ports. The flexible shafts may be looped around the handle and secured to the instrument clip such that the instrument control handles are positioned distally of the clip. Such a configuration may allow for the user to grip onto the handle and simultaneously manipulate the distal end effectors of the tools via the control handles located immediately adjacent to the user's hand.

Another method for facilitating instrument management utilizes forming rigid portions of the instrument shafts. The elongate shaft is generally configured as a flexible length so as to traverse through the elongate body and within the patient body via endoluminal pathways. A portion of the elongate shaft extending between the handle and flexible length may be configured as a rigid section and may include a rigid sleeve made, e.g., from stainless steel or some other rigid metal or polymer, which is formed over the portion of the shaft extending from handle. Alternatively, the rigid portion may be formed integrally with the elongate shaft, e.g., as a section reinforced by woven metallic braids or inserts. In use, the flexible length of the elongate shaft may be advanced through a tool port and through the handle assembly. The rigid section extending from the handle may be advanced at least partially into the tool port such that the handle is supported or held in a linear configuration relative to the tool port and handle assembly by the rigid section.

The interface between the rigid portion(s) of the instrument shaft(s) and the straight sections of the tool port(s) provided in the handle assembly provides the operator with the ability to slidably dock the instruments within the endoluminal access device. The slidable docking interface provides several benefits. For example, the operator is able to release the instrument to use his hand for other purposes without having the instrument drop or flop downward, as would be the case with a flexible shafted instrument. In addition, the slidable docking interface facilitates instrument management using only a single support arm for the endoluminal access device, rather than requiring separate support for each instrument inserted into the device. Further, rigid shafted instruments provide improved force transmission and the slidable docking interface reduces or eliminates the possibility that an exposed shaft will bend or buckle. Still further, having a substantially straight tool port lumen in the handle assembly retains the ability to use flexible shafted instruments, if desired. Finally, having a substantially straight tool port lumen in the handle assembly facilitates insertion of instruments having longer rigid working lengths and/or larger shaft diameters. For example, a typical endoscope has an instrument channel with an inlet having a 45 degree bend. All tools used in the channel must be sufficiently flexible to pass the 45 degree bend. Having a substantially straight lumen provides the ability to use many instruments that could not be used through the instrument channel of a conventional endoscope.

Another variation of the instrument management system includes the provision of a flexible joint or flexible section of the instrument shaft between the handle and a rigid proximal section of the shaft. The flexible joint/section allows the handle to be flexed away from other instruments but retain sufficient rigidity that the handle does not droop. In this manner, the instrument handles are able to be flexed apart to prevent or reduce clashing.

### BRIEF DESCRIPTION OF THE DRAWINGS

FIGS. 1A and 1B shows assembly and end views, respectively, of an endoluminal tissue manipulation system and examples of the various endoluminal instruments which may be advanced therethrough.

FIG. 2 shows the endoluminal manipulation system of FIG. 1A disassembled into its separate instrument components for illustrative purposes.

FIGS. 3A to 3C illustrate side views of a tissue manipulation assembly operable via a launch tube member which may be advanced through the endoluminal system.

FIG. 4 illustrates one configuration for device management between a primary operator and at least a secondary operator.

FIGS. 5A and 5B show top and perspective views, respectively, of a device management system utilizing a control umbilicus having one or more control openings.

FIGS. 6A and 6B show another configuration for instrument positioning and management in perspective and top views, respectively, of an articulatable tray for use by a primary or secondary operator or another person.

FIGS. 7A and 7B illustrate perspective and top views of another configuration utilizing an articulatable support arm having one or more separate instrument booms for supporting each tool.

FIG. 8 illustrates a perspective view of another configuration utilizing an articulatable support arm having one or more independently manipulatable instrument booms attached thereto.

FIG. 9 illustrates yet another device management support platform configured as a curved or arched platform positionable over a patient body.

FIGS. 10A to 10C illustrate perspective and top views of an angled or curved support arm which may be used to restrain a patient's head during endoluminal procedures where instruments are passed trans-esophageally.

FIGS. 11A and 11B illustrate perspective and top views, respectively, of a stiffened multi-instrument support arm having one or more angled or curved support channels projecting therefrom.

FIGS. 12A and 12B illustrate perspective and top views, respectively, of another stiffened multi-instrument support arm having a straight tubular member and one or more angled or curved support channels.

FIGS. 13A to 13C illustrate perspective, top, and end views, respectively, of another variation for a pivoting multi-instrument support having a fanned or angled lumen enclosure.

FIGS. 13D and 13E show top views illustrating examples for altering the entry lumen angle of the individual instrument ports.

FIGS. 13F and 13G show perspective views of a manifold supporting a pair of elongated docking sections that is attachable to the proximal end of an endoluminal access device.

FIG. 14 shows a perspective view of a handle assembly having a detachable instrument clip which may secure one or more flexible shafts inserted through the tool ports.

FIGS. 15A to 15C illustrate side views of an instrument management system utilizing rigid portions of an instrument shaft for providing support to the instrument projecting from a handle assembly.

FIGS. 16A and 16B show end and top views, respectively, of tool ports having tapered entries for facilitating the insertion of instruments therethrough.

FIGS. 17A and 17B show perspective views of a belt or harness which may be worn by an operator to temporarily hold and manage multiple instruments.

FIGS. 18A and 18B show exploded and perspective views of a rotating clamp adapted to be attached to an endoluminal access device.

FIGS. 19A and 19B show an endoscopic device having a straight, elongated docking lumen formed in the handle, and an instrument having a rigid shaft section near its proximal end.

FIGS. 20A and 20B show top views of an endoscopic device handle assembly having a plurality of instruments extending from its proximal end.

### DETAILED DESCRIPTION OF THE INVENTION

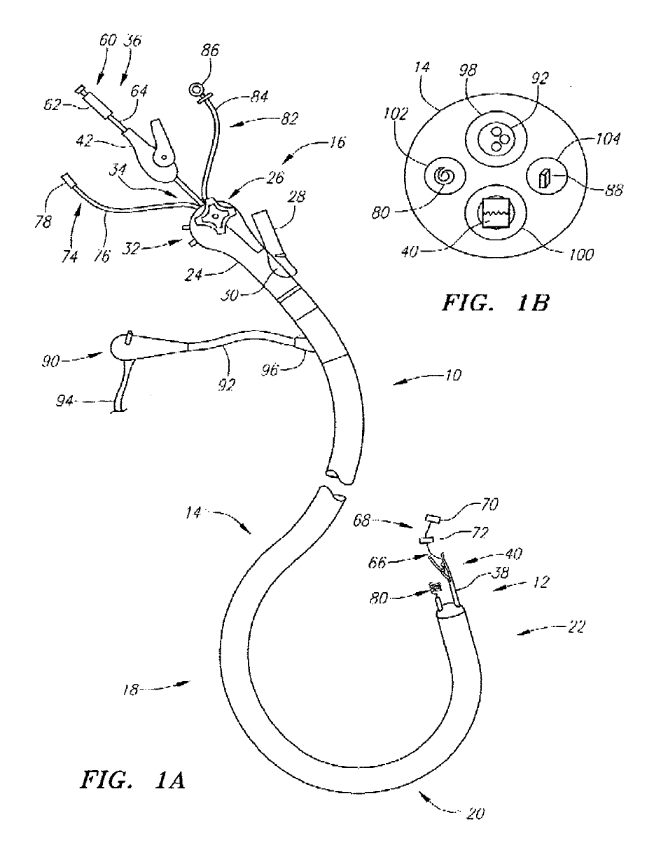

With reference to FIG. 1A, the endoluminal tissue manipulation system 10 as described herein may comprise, at least in part, a distal end effector assembly 12 disposed or positionable at a distal end of a flexible and elongate body 14. Examples of the tissue manipulation system 10 are described in further detail in U.S. Pat. Pub. No. 2005/0272977 A1, which is incorporated herein by reference in its entirety. Additional examples of endoscopic access devices and systems incorporating such devices are described in further detail in U.S. patent application Ser. No. 12/061,591, filed Apr. 2, 2008, which is also incorporated herein by reference in its entirety. A handle assembly 16 may be connected to a proximal end of the elongate body 14 and include a number of features or controls 26 for articulating and/or manipulating both the elongate body 14 and/or the distal end effector assembly 12.

As shown, the system 10 may comprise a number of various instruments and devices utilized in various combinations with one another to effect any number of different procedures. Accordingly, each of the instruments and devices may require manipulation or some degree of handling by the practitioner.

The elongate body 14 may optionally utilize a plurality of locking or lockable links nested in series along the length of the elongate body 14 which enable the elongate body 14 to transition between a flexible state and a rigidized or shape-locked configuration. Details of such a shape-lockable body may be seen in further detail in U.S. Pat. Nos. 6,783,491; 6,790,173; and 6,837,847, each of which is incorporated herein by reference in its entirety. Alternatively, elongate body 14 may comprise a flexible body which is not rigidizable or shape-lockable but is flexible in the same manner as a conventional endoscopic body, if so desired. Additionally, elongate body 14 may also incorporate additional features that enable any number of therapeutic procedures to be performed endoluminally. Elongate body 14 may be accordingly sized to be introduced per-orally. However, elongate body 14 may also be configured in any number of sizes, for instance, for advancement within and for procedures in the lower gastrointestinal tract, such as the colon.

Elongate body 14, in one variation, may comprise several controllable bending sections along its length to enable any number of configurations for the elongate body 14. Each of these bending sections may be configured to be controllable separately by a user or they may all be configured to be controlled simultaneously via a single controller. Moreover, each of the control sections may be disposed along the length of elongate body 14 in series or they may optionally be separated by non-controllable sections. Moreover, one, several, or all the controllable sections (optionally including the remainder of elongate body 14) may be rigidizable or shape-lockable by the user.

In the example of endoluminal tissue manipulation system 10, elongate body may include a first articulatable section 18 located along elongate body 14. This first section 18 may be configured via handle assembly 16 to bend in a controlled manner within a first and/or second plane relative to elongate body 14. In yet another variation, elongate body 14 may further comprise a second articulatable section 20 located distal of first section 18. Second section 20 may be configured to bend or articulate in multiple planes relative to elongate body 14 and first section 18. In yet another variation, elongate body 14 may further comprise a third articulatable section 22 located distal of second section 20 and third section 22 may be configured to articulate in multiple planes as well, e.g., 4-way articulation, relative to first and second sections 18, 20.

As mentioned above, one or each of the articulatable sections 18, 20, 22 and the rest of elongate body 14 may be configured to lock or shape-lock its configuration into a rigid set shape once the articulation has been desirably configured. Detailed examples of such an apparatus having one or multiple articulatable bending sections which may be selectively rigidized between a flexible configuration and a shape-locked configuration may be seen, e.g., in U.S. Pat. Pub. Nos. 2004/0138525 A1, 2004/0138529 A1, 2004/0249367 A1, and 2005/0065397 A1, each of which is incorporated herein by reference in its entirety. Although three articulatable sections are shown and described, this is not intended to be limiting as any number of articulatable sections may be incorporated into elongate body 14 as practicable and as desired. Moreover, one or multiple sections may be comprised of a series of nested-links which allow the one or more sections 18, 20, 22 to be articulated or deflected relative to one another along their lengths and optionally rigidized to conform and hold any particular shape.

Handle assembly 16 may be attached to the proximal end of elongate body 14 via a permanent or releasable connection. Handle assembly 16 may generally include a handle grip 24 configured to be grasped comfortably by the user and an optional rigidizing control 28 if the elongate body 14 and if one or more of the articulatable sections are to be rigidizable or shape-lockable. Rigidizing control 28 in this variation is shown as a levered mechanism rotatable about a pivot 30. Depressing control 28 relative to handle 24 may compress the internal links within elongate body 14 to thus rigidize or shape-lock a configuration of the body while releasing control 28 relative to handle 24 may in turn release the internal links to allow the elongate body 14 to be in a flexible state. Further examples of rigidizing the elongate body 14 and/or articulatable sections may again be seen in further detail in U.S. Pat. Pub. Nos. 2004/0138525 A1, 2004/0138529 A1, 2004/0249367 A1, and 2005/0065397 A1, incorporated above by reference. Although the rigidizing control 28 is shown as a lever mechanism, this is merely illustrative and is not intended to be limiting as other mechanisms for rigidizing an elongate body, as generally known, may also be utilized and are intended to be within the scope of this disclosure.

Handle assembly 16 may further include a number of articulation controls 26, as described in further detail below, to control the articulation of one or more articulatable sections 18, 20, 22. Handle 16 may also include one or more ports 32 for use as insufflation and/or irrigation ports, as so desired.

Furthermore, one or more various instruments may be passed through elongate body 14 for deployment through distal end 12 by introducing the instruments through one or more corresponding tool ports 34 located in handle assembly 16. As mentioned above, a number of different endoscopic and/or endoluminal instruments having a flexible body may be delivered through system 10 to effect any number of endoluminal procedures.

One example of such an instrument may include an endoluminal tissue manipulation and securement assembly 36, as described in further detail below, which may be introduced into system 10 via instrument lumen 100, as shown in the end view of distal end 12 in FIG. 1B. Any number of additional instruments may also be inserted through the system 10. An example of such an instrument includes an elongate tissue engagement tool 74 having an elongate flexible shaft 76 with a removable handle or grip 78 located on its proximal end. The tissue engagement tool 74 may be positioned within an instrument lumen 102 adjacent to instrument lumen 100. The distal end of flexible shaft 76 may include a rotatable helical tissue engager 80 used to temporarily engage and manipulate tissue. The helical tissue engager 80 may further include a number of visual indications or markers near or at the distal end of flexible shaft 76. Examples of tissue engagement tool 74 are described in further detail in U.S. patent application Ser. No. 11/303,521 filed Dec. 16, 2005, which is incorporated herein by reference in its entirety.

In use, tissue manipulation assembly 40 and helical tissue engager 80 may be advanced distally out from elongate body 14 through their respective lumens 100, 102. Tissue engager 80 may be advanced into contact against a tissue surface and then rotated via its proximal handle 78 until the tissue is engaged. The engaged tissue may be pulled proximally relative to elongate body 14 and tissue manipulation assembly 40 may be actuated via its proximally located handle into an open expanded jaw configuration for receiving the engaged tissue.

Additional instruments may also be introduced through elongate body 14, such as conventional endoscopic instruments including graspers, scissors, needle knives, snares, etc., through a corresponding instrument lumen 104. In one example, an endoscopic instrument 82 having a flexible shaft 84 with a manipulatable handle or control 86 at its proximal end and a scissor mechanism 88 at its distal end may be introduced through the elongate body 14 for performing tasks such as cutting of tissue and/or sutures.

To endoluminally visualize procedures and tissue regions of interest, an endoscope or imaging system 90 having a flexible shaft 92 may be introduced into the elongate body 14 via a side port, e.g., Y-Port 96, located along the elongate body 14 and distal to handle assembly 16, as shown in FIG. 1A. Flexible shaft 92 may be advanced through visualization lumen 98 such that its distal end is advanced distally of the elongate body distal end 12 or it may be parked at the terminal opening of the visualization lumen 98 for providing imaging of a procedure. Although shown as an endoscope 90 in this illustration, other variations may include an imaging chip such as a CCD imager integrated into the distal end 12 of elongate body 14. A cable 94 extending from endoscope 90 may be connected to a processor and monitor (not shown) for providing the images.

Endoscope 90 may be introduced directly through handle assembly 16 in other variations; however, positioning the imaging system 90 through a distally located Y-Port 96 relative to handle assembly 16 may allow for a longer length of the shaft 92 to be introduced through visualization lumen 98 into the patient body. As elongate body 14 is advanced into the patient body, e.g., per-orally and into the stomach, the Y-Port 96 remains outside the patient body.

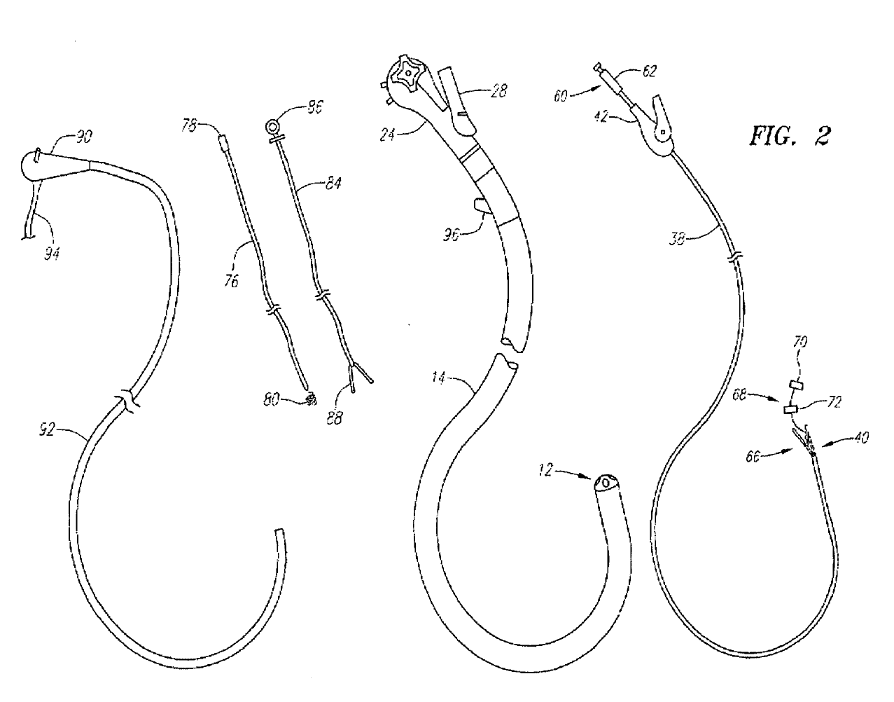

FIG. 2 shows endoluminal manipulation system 10 disassembled into its separate instrument components for illustrative purposes. As seen, the handle 42 of tissue manipulation assembly 40 and its flexible shaft 38 may be removed from elongate body 14. Removable needle deployment assembly 60 with its needle assembly control or housing 62 and its elongate shaft extending through flexible shaft 38 and terminating in needle assembly 66 may also be removed from elongate body 14. Also shown is anchor assembly 68 comprising, e.g., distal tissue anchor 70 and proximal tissue anchor 72, which may be deployed from needle assembly 66 through flexible shaft 38.

Also shown is helical tissue engager 80 disposed upon flexible shaft 76 and endoscopic instrument 88, e.g., endoscopic scissors, disposed upon flexible shaft 84, removed from elongate body 14 and handle assembly 16. Further shown is endoscope 90 with endoscope shaft 92 removed from Y-Port 96.

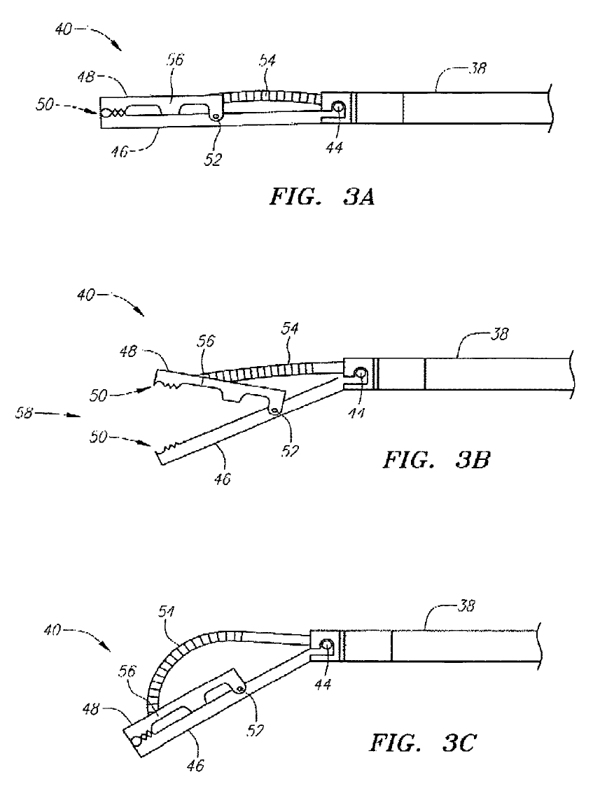

As mentioned above, tissue manipulation assembly 40 is further described in detail in U.S. patent application Ser. No. 11/070,863 filed Mar. 1, 2005 and published as U.S. Pat. Pub. 2005/0251166 A1. An illustrative side view of one example is shown in FIG. 3A, which shows assembly 36. The assembly 36 generally comprises a flexible catheter or tubular body 38 which may be configured to be sufficiently flexible for advancement into a body lumen, e.g., transorally, percutaneously, laparoscopically, etc. Tubular body 38 may be configured to be torqueable through various methods, e.g., utilizing a braided tubular construction, such that when handle 42 is manipulated and/or rotated by a practitioner from outside the patient's body, the longitudinal and/or torquing force is transmitted along body 38 such that the distal end of body 38 is advanced, withdrawn, or rotated in a corresponding manner.

Tissue manipulation assembly 40 is located at the distal end of tubular body 38 and is generally used to contact and form tissue folds, as mentioned above. FIG. 3B shows an illustrative detail side view in which tissue manipulation assembly 40 may be seen connected to the distal end of tubular body 38 via a pivotable coupling 44. Lower jaw member 46 extends distally from the pivotable coupling 44 and upper jaw member 48, in this example, may be pivotably coupled to lower jaw member 46 via jaw pivot 52. The location of jaw pivot 52 may be positioned at various locations along lower jaw 46 depending upon a number of factors, e.g., the desired size of the “bite” or opening for accepting tissue between the jaw members, the amount of closing force between the jaw members, etc. One or both jaw members 46, 48 may also have a number of protrusions, projections, grasping teeth, textured surfaces, etc., 50 on the surface or surfaces of the jaw members 46, 48 facing one another to facilitate the adherence of tissue between the jaw members 46, 48.

Launch tube 54 may extend from handle 42, through tubular body 38, and distally from the end of tubular body 38 where a distal end of launch tube 54 is pivotally connected to upper jaw member 48 at launch tube pivot 56. A distal portion of launch tube 54 may be pivoted into position within a channel or groove defined in upper jaw member 48, to facilitate a low-profile configuration of tissue manipulation assembly 40. When articulated, either via launch tube 54 or other mechanism, as described further below, jaw members 46, 48 may be urged into an open configuration to receive tissue in jaw opening 58 between the jaw members 46, 48.

Launch tube 54 may be advanced from its proximal end at handle 42 such that the portion of launch tube 54, which extends distally from body 38, is forced to rotate at hinge or pivot 56 and reconfigure itself such that the exposed portion forms a curved or arcuate shape that positions the launch tube opening perpendicularly relative to upper jaw member 48, as shown in FIG. 3C. Launch tube 54, or at least the exposed portion of launch tube 54, may be fabricated from a highly flexible material or it may be fabricated, e.g., from Nitinol tubing material which is adapted to flex, e.g., via circumferential slots, to permit bending.

Once the tissue has been engaged between jaw members 46, 48, a needle deployment assembly 60 may be urged through handle 42 and out through launch tube 54 by introducing needle deployment assembly 60 into the handle 42 and through tubular body 38 such that the needle assembly 66 is advanced from the launch tube and into or through approximated tissue. The needle deployment assembly 60 may pass through lower jaw member 46 via needle assembly opening defined in lower jaw member 46 to pierce through the grasped tissue. Once the needle assembly 66 has been passed through the engaged tissue, a distal and proximal tissue anchor 70, 72 of the anchor assembly 68 may be deployed or ejected on one or opposing sides of a tissue fold for securing the tissue.

Anchor assembly 68 is normally positioned within the distal portion of tubular sheath 64 which extends from needle assembly control or housing 62. Once the anchor assembly 68 has been fully deployed from sheath 64, the spent needle deployment assembly 60 may be removed from assembly 36 and another needle deployment assembly may be introduced without having to remove assembly 36 from the patient. The length of sheath 64 is such that it may be passed entirely through the length of tubular body 38 to enable the deployment of needle assembly 66 into and/or through the tissue.

Because of the number of different instruments and the different types of tools which may be utilized in endoluminal tissue manipulation system 10, tool or instrumentation management is one consideration for the practitioner or practitioners to facilitate efficient surgical and/or endoscopic procedures when performed upon a patient. Additionally, the division of responsibility for instrumentation management between one or more practitioners is highly desirable to ensure patient safety and procedure facilitation.

FIG. 4 illustrates one configuration for device management between a primary operator and at least a secondary operator. As shown, a first set of instruments 110, including operation of the elongate body 14 via handle 24 and transitioning elongate body 14 between its rigid and flexible states via control 28 and operation of tissue manipulation assembly 40 via handle 42 may be controlled via a primary operator. A second set of instruments 112, including operation and control of the endoscope or imaging system 90 through Y-Port 96 and operation of the one or more tools such as manipulating control 86 and handle 78, may be controlled via a secondary operator who may be positioned along side or proximate to the primary operator. Accordingly, the second set of instruments 112 may be positioned away from the first set of instruments 110 to facilitate procedures.

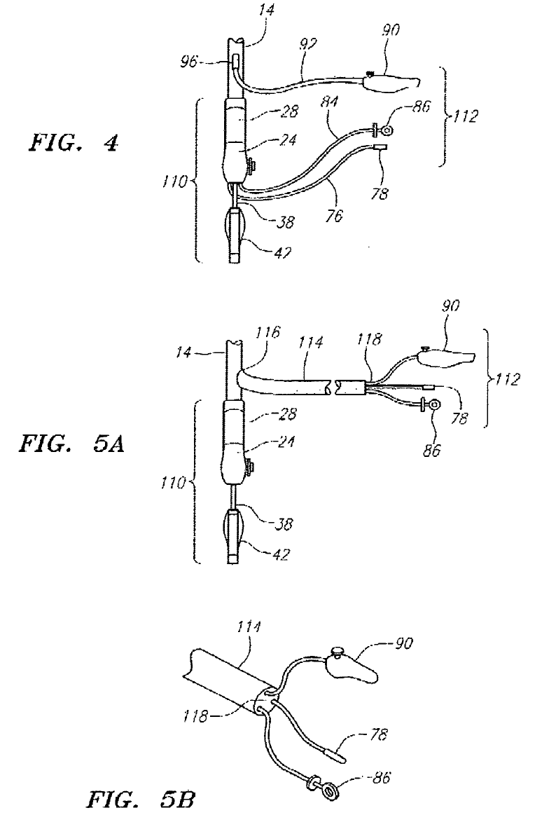

Another configuration may include the use of a control bundle or umbilicus 114 which may be connected to handle 24 or to elongate body 14 via an umbilicus port 116, as shown in FIG. 5A. Control umbilicus 114 may be a bundle of individual lumens or a flexible tubular member having individual lumens routed therethrough which connect to corresponding lumens routed through elongate body 14. A terminal end of umbilicus 114 may define a number of umbilicus control openings 118 for entry and passage of the second set of instruments 112, as shown in FIG. 5B. Use of a single umbilicus 114 extending from elongate body 14 or handle 24 may facilitate the handling and positioning of multiple instruments for a secondary operator.

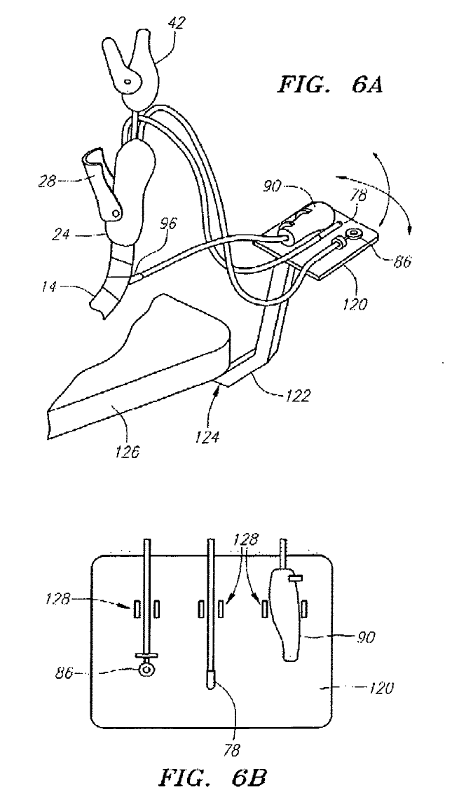

Yet another configuration for instrument positioning and management is shown in the perspective view of FIG. 6A, which illustrates the use of an articulatable tray 120 for use by the primary or secondary operator or another person. Tray 120 may be a stand-alone tray or one attached via attachment 124 to a patient bed or operating table 126 via an articulatable support arm 122. Support arm 122 may be pivoted or translated relative to table 126 in any number of positions, as shown by the arrows, to facilitate use as a support for the second set of instruments 112.

Tray 120 may have a surface upon which one or more holders 128 may be positioned to temporarily hold onto or retain a corresponding tool. For instance, as shown in the top view of FIG. 6B, the handle of endoscope 90, as well as controls 78, 86 of the corresponding instruments may be removably held via holders 128. Holders 128 may conform to any number of shapes and configurations, e.g., clips, biased retainers, etc., for temporarily retaining the corresponding instrument. Use of tray 120 may further allow one or more operators to control each individual instrument without having to individually remove and articulate each instrument.

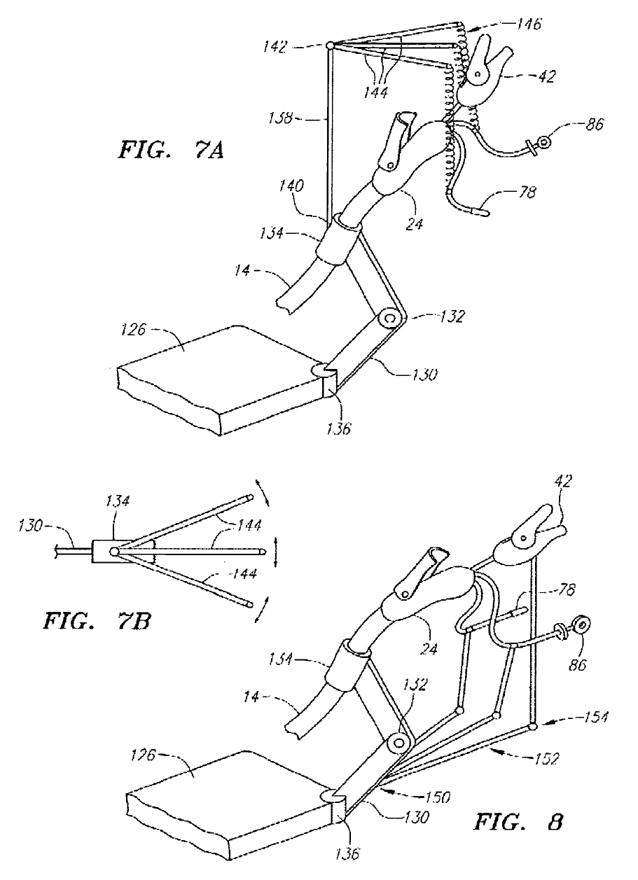

In yet another configuration, a single support arm 130 may be utilized to manipulate and manage the entire system. As illustrated in FIG. 7A, support arm 130 may be attached to table 126 with a pivotable or static connection 136; alternatively, support arm 130 may be a stand-alone member. In either case, support arm 130 may be one or more articulatable pivots or joints 132 which allow for support attachment 134 to be moved and held in different arrangements. Support attachment 134 may be configured to securely hold elongate body 14 therethrough, e.g., through an annular support or C-clamp type support which is configured to temporarily lock onto elongate body 14. Support attachment 134 may further have an instrument support arm 138 pivotably connected at a first end via pivot 140 to support attachment 134. A second end of instrument support arm 138 may be further connected via pivot 142 to one or more instrument booms 144 which project or extend over handle assembly 24. Each of the instrument booms 144 may be attachable either directly or via extendable and/or retractable instrument supports 146 to a corresponding instrument extending from elongate body 14 or handle 24.

As shown in the top view of the instrument booms 144 in FIG. 7B, each of the booms 144 may be independently movable or articulatable to position the boom 144 over a corresponding instrument. Moreover, the instrument supports 146 may be comprised of a biased member or spring so that the supported instruments, e.g., instrument handles 78, 86, may be used or held by the operator at any desired angle relative to the handle 24 and then released while supported by the instrument support 146. Additionally, the multiple pivotable connections between each of the members may allow for multiple degrees-of-freedom in managing and positioning each of the instruments relative to the patient. Also, the use of multiple degree-of-freedom instrument supports may further allow for a single operator, or at least a minimal number of support personnel, to control the entire system.

Another variation for device management is shown in the perspective view of FIG. 8, which illustrates one or more instrument support arms 152 optionally having one or more pivotable connections 154 attached directly to support arm 130 at attachment location 150. Alternatively, support arms 152 may be attached directly to table 126 or to a separate stand-alone support. In either case, support arms 152 may have multiple degrees-of-freedom to allow for positioning of each supported corresponding instrument in any various configuration relative to handle 24 and the patient.

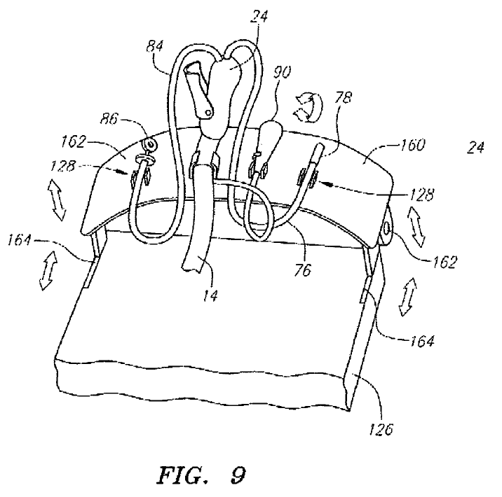

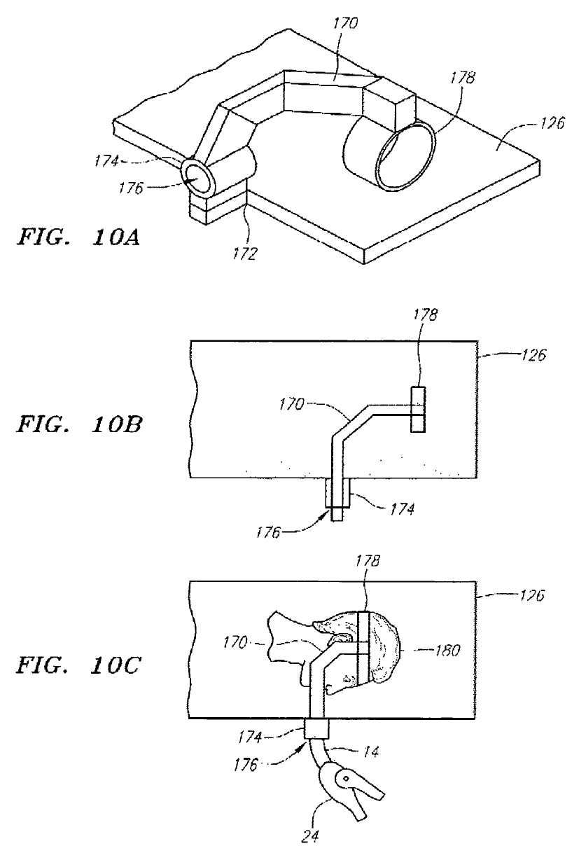

FIG. 9 illustrates yet another device management support platform 160 which may be attached directly to table 126, as shown, or which may be configured as a separated stand-alone support which is positionable over or adjacent to table 126. In this example, platform 160 may be configured as a curved or arched platform which is raised over table 126 to allow for elongate body 14 to easily access the patient body, e.g., through a patient's mouth. Platform 160 may be attached to table 126 via pivotable joints 162 which allow for angular adjustment between a horizontal and a vertical position of platform 160, as indicated by the rotating arrow. Platform 160 may also be translatably connected to table 126 via a slidable connection 160 to allow for sliding adjustment along table 126 as well as to allow for height adjustment relative to the patient body, as indicated by the translational arrows.

Each of the instruments and elongate body 14 may be temporarily attached or connected via one or more corresponding attachments 128 positioned along platform 160. As arched platform 160 is rotated or angled and/or adjusted in height or along table 126, each of the instruments will accordingly move along with platform 160. Use of a single platform 160 may allow for a single operator or minimum number of operators to utilize the system.

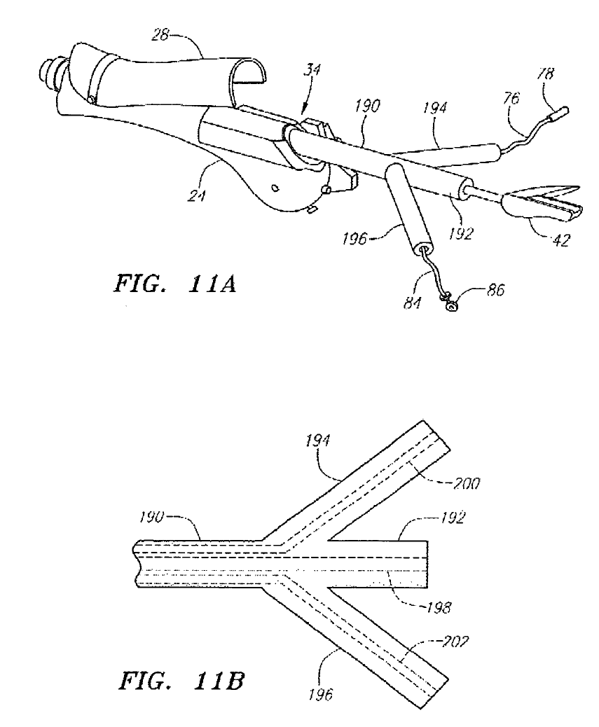

Another platform to facilitate use of the system with a patient is shown in the perspective and top views of FIGS. 10A and 10B, respectively. The angled or curved support arm 170 may be attached to table 126 via an adjustable attachment point 172 along the side of table 126. An opening 176 may be defined through a portion of support arm 170 through which the elongate body 14 may be passed prior to accessing the patient. The elongate body 14 may be attached locked temporarily within opening 176 during procedures performed upon patients. The elongate body 14 may be passed through opening 176 such that it passes directly into a patient's mouth for trans-esophageal entry into the patient body. Restraining strap 178 may be connected to a distal portion of support arm 170 such that when a patient is positioned upon table 126 in a lateral position, restraining strap 178 may be gently secured to patient's head 180, as shown in FIG. 10C, to maintain in a stable manner an orientation of the patient's head relative to opening 176, particularly when elongate body 14 has been inserted through the patient's mouth.

In several of the embodiments described above, the endoluminal access system is supported by a single support arm or support stand that is attached to the handle or other portion of the system. Other support arms or stands are suitable for use in alternative embodiments. For example, conventional surgical or laparoscopic stands typically include rigid linkages connected by ball joints that are tightened by application of a central control knob. Two linkages having ball joint terminations provide an effectively unrestricted range of motion. Typically, the base of the first linkage terminates in a feature that is adapted to clamp to the bed rail, and the distal end of the second linkage terminates in a clamp adapted to attach to a surgical or laparoscopic instrument. These stands may be actuated mechanically, electromechanically, pneumatically, or otherwise under manual, foot, or voice control.

Several of the instrument management system embodiments described herein facilitate use of the endoluminal access system by the operator in either a “hands on tools” mode with the system retained in the stand or support arm, or a “hand on scope/hand on tool” mode in which the operator holds the handle 24 in one hand and an instrument with the other hand. Those skilled in the art will recognize that the “hands on tools” mode corresponds generally with the manner in which laparoscopic procedures are typically performed, while the “hand on scope/hand on tool” mode corresponds generally with the manner in which endoscopic procedures are performed. Each of these modes of use are facilitated using the instrument management systems described herein. For example, many surgical instrument holders are configured to clamp onto the shaft of a 5 mm or 10 mm instruments. By providing a 5 mm or 10 mm cylindrical post on the handle 24 of an endoluminal access system, the handle 24 may be selectively clamped onto and removed from the instrument holder by the operator. In this way, the operator can simply place the post in the holder and lock it in place to use the system in a “hands on tools” mode, or remove it from the holder and use the system in a “hand on handle/hand on tool” mode.

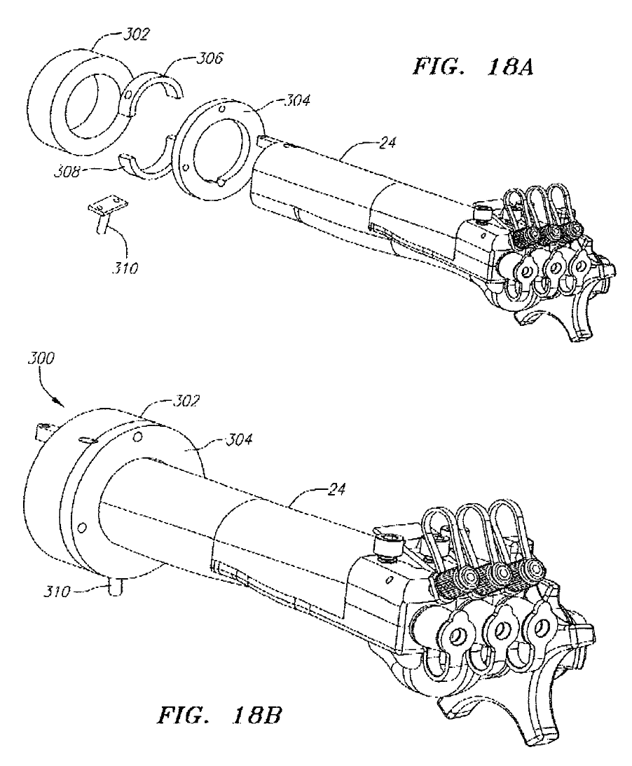

Aside from or in addition to table-mounted or stand-alone instrument supporting members, additional instrument management systems may be employed which a single operator or user may utilize. One example is shown in FIGS. 18A and 18B, which show perspective views of a handle 24 and a rotating clamp mechanism 300 that serves as a functional interface between a support arm (e.g., a stand or other holder) and the endoluminal access system. The clamp 300 includes a generally cylindrical housing 302, a backing plate 304, an upper clamp half 306, and a lower clamp half 308. The housing 302 is generally cylindrical in shape, having a central through hole having a size sufficient to allow the handle 24 to pass therethrough. The housing 302 also includes a channel formed on its inner surface and adapted to receive the upper clamp half 306 and lower clamp half 308, each of which has a generally semi-circular shape to facilitate rotational movement within the housing channel. The backing plate 304 is attached to each of the upper clamp half 306 and lower clamp half 308 and the combined unit is fixed to the outer surface of the handle 24. As a result, the handle 24 is allowed to rotate within the clamp housing 302 while being supported by the clamp mechanism 300. A post 310 is attached to the clamp housing 302. The post 310 has a size and shape that facilitates attachment to a clamp or other mechanism contained on the stand, support arm or other mechanism, thereby providing the ability to mount the endoluminal access system on the stand or support arm while providing free rotation of the handle 24 relative to the stand or support arm.

Another instrument management system is shown in FIG. 11A, which shows a perspective view of handle 24 having a multi-instrument support arm 190 extending therefrom. Support arm 190 may generally comprise a stiffened multi-lumen channel having a straight support channel 192 extending proximally and one or more angled or curved support channels 194, 196 projecting at an angle from support arm 190. Although two angled support channels are shown in this illustration, additional support arms may be utilized as practicable and as desired depending upon the number of tools advanced through elongate body 14. In the example, handle 42 of tissue manipulation assembly 40 is positioned through the straight support channel 192 while instrument shafts 76, 84 are positioned through their respective support channels 194, 196.

As shown in the partial cross-sectional view of FIG. 11B, each support channel may have a corresponding separate lumen defined therethrough. For instance, straight support channel 192 may have instrument lumen 198 defined therethrough, while angled support channels 194, 196 may have respective instrument lumens 200, 202 defined therethrough. Because multi-instrument support arm 190 is relatively stiff, e.g., support arm 190 may be comprised of a metal such as stainless steel or a stiffened polymeric material or plastic, support arm 190 may be engaged to handle 24 and used to support and separate its respective instruments leaving the operator to hold a single handle 24 during a procedure.

In an alternative configuration, portions of or the entire support arm 190 is formed of a relatively flexible material, such as a rubber or polymeric material. The flexibility of the support arm 190 allows instruments having relatively rigid shafts to pass through the instrument lumens 198, 200, 202 despite the presence of any non-linear portions of the lumens. For example, the support arm 190 is sufficiently flexible that the support channels 194, 196 are able to flex in response to the rigid instrument shaft as it passes through any non-linear portions of the lumen.

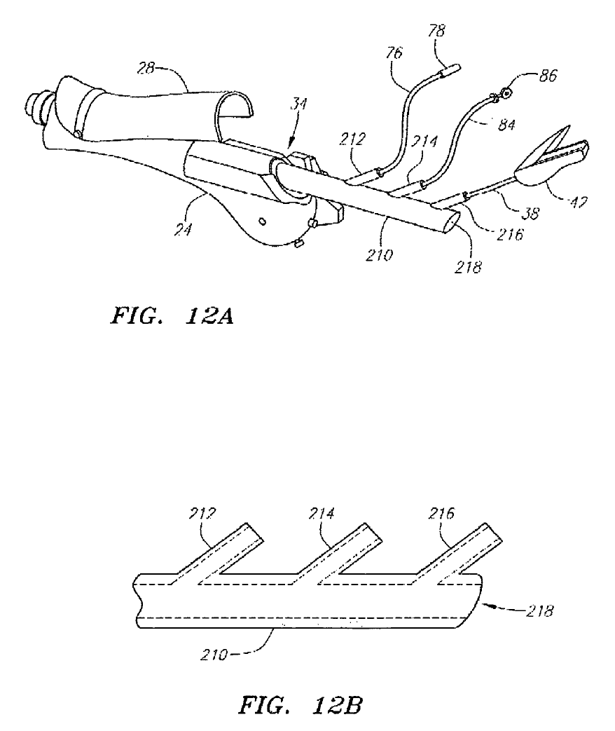

Another example of a multi-instrument support arm 210 is shown in the perspective view of FIG. 12A, which illustrates a straight tubular member 210 which defines a lumen therethrough 218 and having one or more angled or curved support channels 212, 214, 216 each defining an instrument lumen therethrough, as shown in the partial cross-sectional view of FIG. 12B. In an alternative embodiment, each of the support channels 212, 214, 216 provides access to a separate instrument lumen extending through the support arm 210, the handle 24, and the elongate body 14. In this variation, each of the instruments, positioned through each respective channel, may be supported by the support arm 210 and separated for individual control and manipulation. As above, support arm 210 may be made from a stiff material to enable manipulation of handle 24 while support arm 210 supports the various instruments during a procedure.

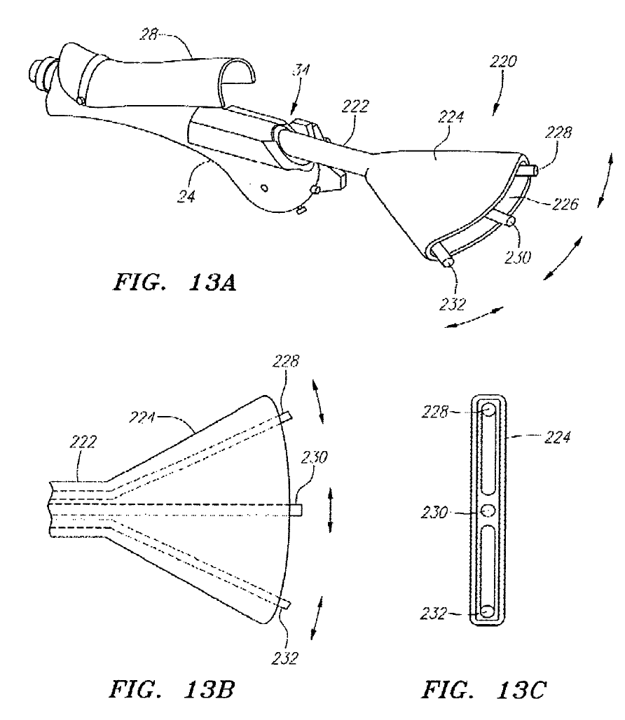

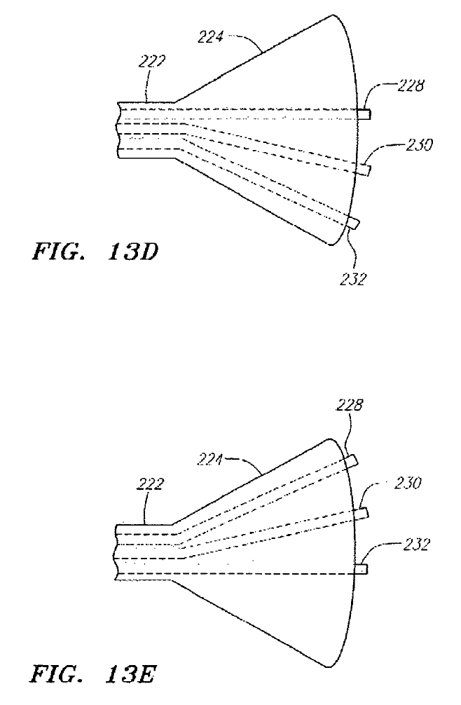

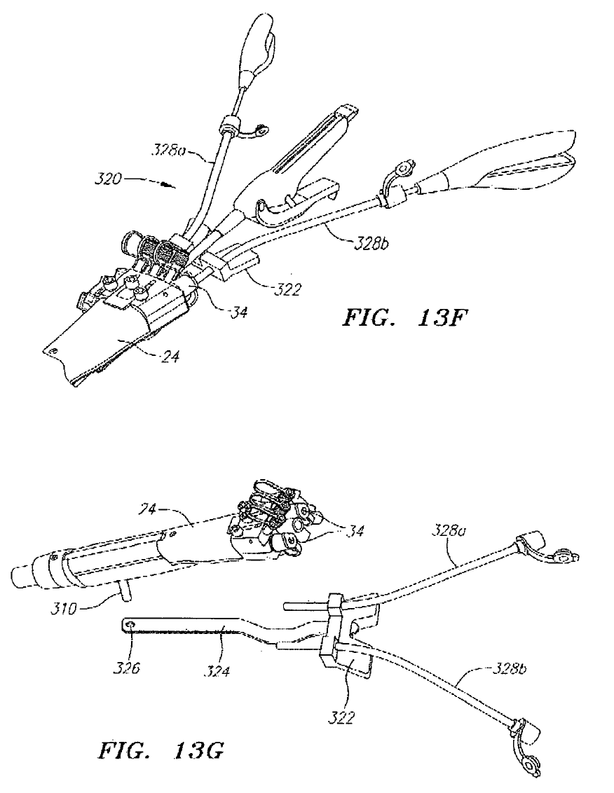

In yet another variation, a pivoting multi-instrument support 220 is illustrated as generally having a support arm 222 with a fanned or angled lumen enclosure 224 extending therefrom, as shown in FIG. 13A. Enclosure 224 may define an open channel 226 within which one or more individual instrument ports 228, 230, 232 may be pivotably positioned, as shown in the top and end views of FIGS. 13B and 13C, respectively. The instruments to be advanced through elongate body 14 may be passed into their respective instrument ports, each of which may be individually pivoted within open channel 226 respect to one another.

FIGS. 13D and 13E show examples of how each individual instrument port 228, 230, 232 may be pivoted into a straightened lumen to facilitate handling or articulation of an individual instrument positioned within a respective port. For instance, as shown in FIG. 13D, instrument port 228 may be pivoted within enclosure 224 to straighten its lumen. If another instrument, which may be positioned within instrument port 232, were to be straightened within enclosure 224, e.g., for withdrawal or advancement, each instrument port may be pivoted within enclosure 224 until the selected port 232 were positioned into its straightened configuration, as shown in FIG. 13E.

Turning to FIGS. 13F and 13G, an alternative multi-instrument support mechanism 320 includes a manifold 322 that is attached to the handle 24 of an endoluminal access system. In the embodiment shown, the manifold 322 includes an elongated tab 324 having a hole 326 that attaches to a post 310 on the handle 24. The manifold 322 supports a plurality of elongated docking sections 328a, 328b, each of which extends from an instrument port 34 of the handle 24. Each docking section 328a, 328b comprises a rigid tube having an elongated straight section adapted to receive a flexible instrument and route the instrument shaft into the respective instrument port 34 and through the handle 24 and elongate body 14 of the endoluminal access system. The docking sections 328a, 328b may optionally include a bend or other feature, such as the bends shown in the embodiment shown in FIGS. 13F and 13G. The bends provide a spread alignment of the instruments retained within the docking sections 328a, 328b to thereby reduce or prevent clashing of the instrument handles. The spread alignment may take several optional forms. For example, all of the instruments retained in the docking sections 328 may be extended an equal length beyond the proximal end of the handle 24 and spread in a single plane or in multiple planes. For illustrative purposes, the system shown in FIGS. 13F and 13G illustrates a spread in a single plane but with a central instrument extended a shorter length from the proximal end of the handle 24. In alternative embodiments, the docking sections 328a, 328b are separately positionable so as to provide the user with a desired spread or orientation.

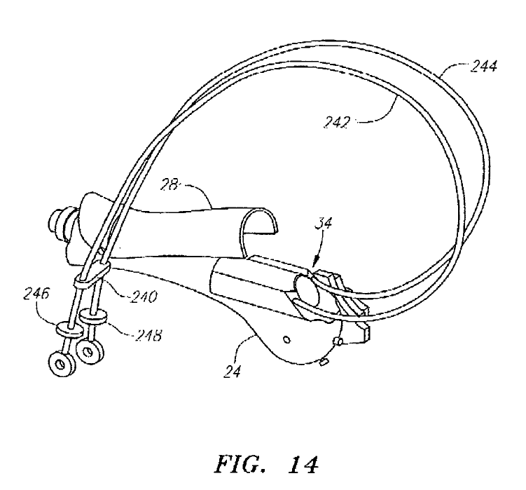

Yet another instrument management system is illustrated in FIG. 14, which shows detachable instrument clip 240 attached to a distal portion of handle 24. Instrument clip 240 may be opened and closed to securely receive the flexible shafts 242, 244 therein. Moreover, the proximal portions of the one or more flexible shafts 242, 244 inserted through tool ports 34 in handle 24 may be looped around handle 24 and secured to instrument clip 240 such that the instrument control handles 246, 248 are positioned distally of clip 240. This configuration may allow for the user to grip onto handle 24 and simultaneously manipulate the distal end effectors of the tools via control handles 246, 248, which are located immediately adjacent to the user's hand. Once a procedure is completed or another instrument is to be loaded within tool port 34, the appropriate tool handle may be detached or removed from instrument clip 240 and the additional instrument handle may be subsequently attached to clip 240.

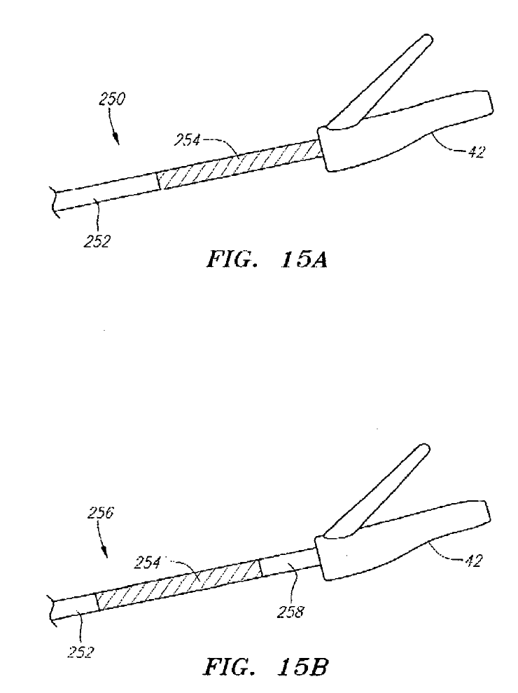

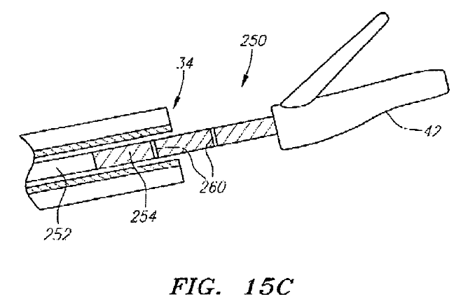

Another method for facilitating instrument management utilizes forming rigid portions of the instrument shafts. An example is shown in the side view of FIG. 15A which illustrates handle 42 and a proximal portion 250 of the elongate shaft of the tissue manipulation assembly 40. The elongate shaft is generally configured as a flexible length 252 so as to traverse through elongate body 14 and within the patient body via endoluminal pathways. A portion of the elongate shaft extending between handle 42 and flexible length 252 may be configured as a rigid section 254. Rigid section 254 may include a rigid sleeve made, e.g., from stainless steel or some other rigid metal or polymer, which is formed over the portion of the shaft extending from handle 42. Alternatively, the rigid portion 254 may be formed integrally with the elongate shaft, e.g., as a section reinforced by woven metallic braids or inserts. Rather than having the rigid section 254 extend directly from handle 42, rigid section 254 may be positioned between two flexible lengths 252, 258, as shown in the rigidized elongate body 256 in FIG. 15B.

In use, the flexible length of elongate shaft 252 may be advanced through a tool port 34 and through handle assembly 16. Rigid section 254 extending from handle 42 may be advanced at least partially into tool port 34, as shown in FIG. 15C, such that handle 42 is supported or held in a linear configuration relative to tool port 34 and handle assembly 16 by the rigid section 254. The absence of rigid section 254 from flexible shaft 252 would allow handle 42 to flex and bend relative to tool port 34 in an uncontrolled manner. In the case where a configuration as shown in FIG. 15B is used, rigid section 254 may be positioned to extend from the entry of tool port 34 to provide some support to handle 42 while the proximal flexible section 258 extending between rigid section 254 and handle 42 may still allow for some limited flexibility in moving or articulating handle 42 in a non-linear manner relative to tool port 34 and handle assembly 16.

Additionally, one or more visual markings or indicators 260 may be provided along the length of rigid section 254, as shown in FIG. 15C. These visual indicators 260 may correspond to the depth which the tissue manipulation assembly 40 has been inserted into the patient body or the length which tissue manipulation assembly 40 has been advanced past the distal end of the rigidizable elongate body 14 within a body lumen of a patient.

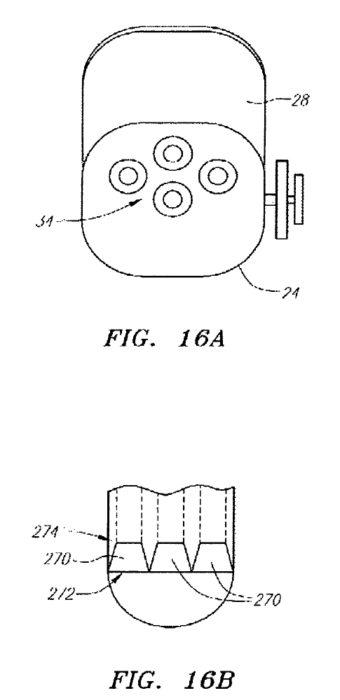

In addition to the various device and instrument management tools and systems described above, tool ports 34 in handle assembly 16 may also be configured to facilitate device management. As shown in the end and top views of handle assembly 16 in FIGS. 16A and 16B, respectively, the entry to tool ports 34 may be configured as a tapered instrument port 270. Tools and instruments may be inserted through the enlarged entry 272 and guided into the narrower tool lumen 274 by the narrowing tapered surface of port 270.

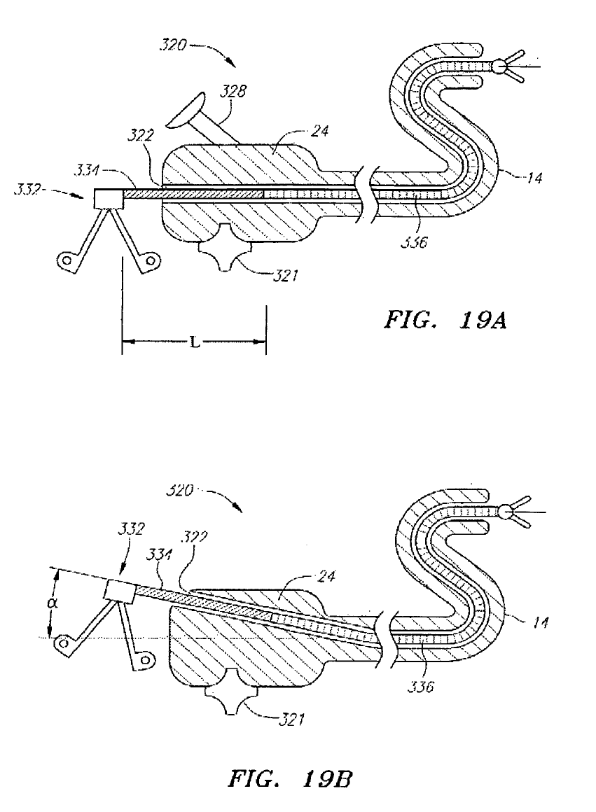

Several of the features of the tools and systems described above in relation to FIGS. 11-13 and 15-16 are further described in relation to FIGS. 19A and 19B, which illustrate the slidable docking feature of an endoscopic access device and a flexible instrument. Referring to FIG. 19A, an endoscopic access device 320 is shown, the device having a handle 24 with an eyepiece 328 and steering controls 321. The device includes an instrument channel 322 extending through the handle 24 that is elongated and substantially straight through at least a proximal section. The instrument 332 includes a shaft having a substantially rigid proximal section 334 and a substantially flexible distal section 336. As described above, the slidable docking interface provided between the instrument channel 322 and the rigid proximal shaft 334 allows the operator to release the instrument, upon which the instrument will remain stably docked within the handle 24 of the access device 320. In several embodiments, the length L of the rigid proximal section 334 of the instrument shaft is no longer than the rigid length of the instrument channel 322 so as not to interfere with the flexibility of the flexible section of the endoscopic access device 320 when the instrument shaft is inserted into the device to its intended extent. The length L of the rigid proximal section 334 should, however, be sufficient to provide additional overlap so that slidable docking occurs (i.e., no backing out to the flexible shaft section 336) during normal operation of the instrument.

As shown in FIG. 19B, the elongated and substantially straight section of the instrument channel 322 extending through the handle 24 need not be in line with longitudinal axis of the flexible section 14 of the endoluminal access device. In the embodiment shown in FIG. 19B, the docking section of the instrument channel 322 is inclined at an angle α relative to the longitudinal axis of the flexible section 14. The flexible portion of the instrument shaft 336 is sufficiently flexible to accommodate the bend created by the differential.

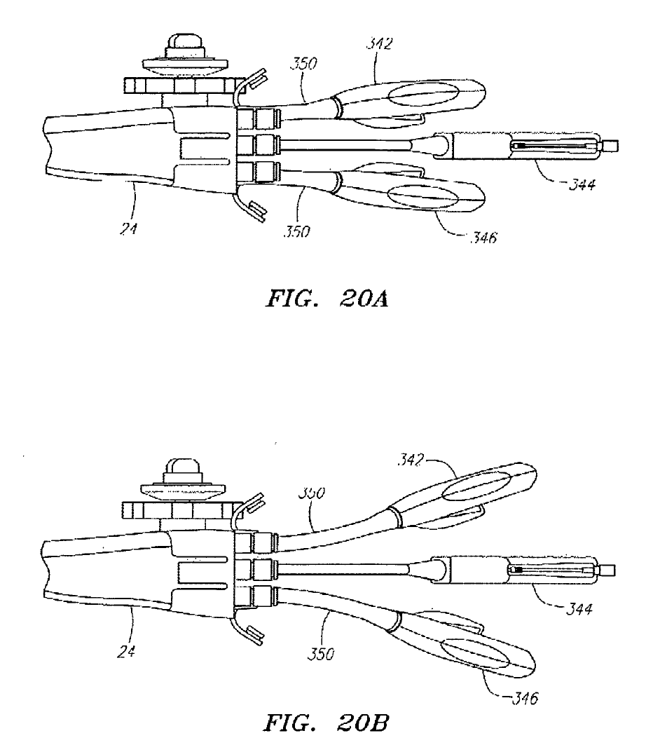

In addition to the other instrument management tools and systems described herein, another mechanism for reducing or eliminating clashing of instrument handles is shown in FIGS. 20A and 20B. An endoscopic device handle 24 includes a plurality of instruments 342, 344, 346 extending from a plurality of instrument ports 34 located on the proximal end of the handle. As shown in FIG. 20B, two of the instruments include a flexible joint 350 located adjacent to the instrument handle between the handle and the rigid portion 334 of the instrument shaft. The flexible joint 350 are sufficiently flexible to allow the handle to be bent away from handles of other instruments received in the device while retaining sufficient rigidity to prevent drooping. In this manner, the handles of adjacent instruments may be flexed apart rather than clashing.

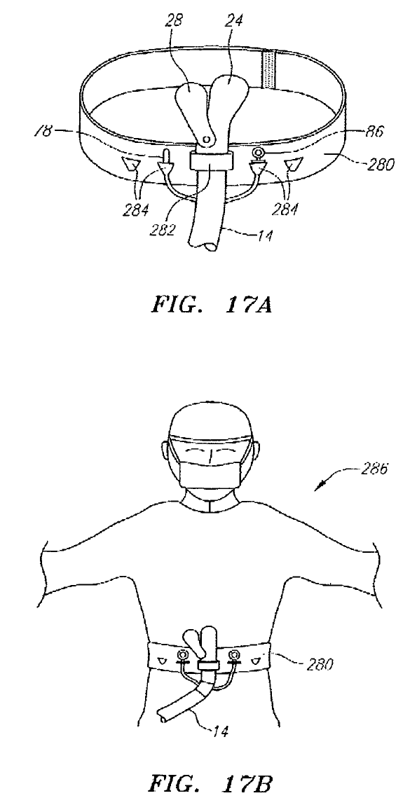

In yet another system for managing the various instruments and tools, a support belt or harness 280 worn by the operator or an assistant may be utilized rather than placing and positioning the instruments upon a stand, tray, or support. As shown in the assembly view of FIG. 17A, one example of such a support belt or harness 280 is illustrated where belt or harness 280 may have one or more instrument holders 282, 284 attached thereto for temporarily holding a corresponding instrument. In use, the physician or operator 286, shown in FIG. 17B, may wear the belt or harness 280 around his/her waist, shoulders, etc. When the operator 286 has finished using a particular tool or is in the process of changing tools, operator 286 may temporarily place the instrument into a corresponding holder 282, 284 on belt or harness 280 thus freeing his/her hands. If so desired, the operator 286 may reuse and replace the instruments as necessary upon belt or harness 280.

The foregoing descriptions of instrument management tools and systems includes descriptions of several components (and embodiments of components) that may be used in a standalone manner or in combination with other components. For example, a preferred embodiment of an instrument management system suitable for use with the endoluminal tissue manipulation system 10 shown in FIG. 1A includes a support stand having a base that is attachable to a bed rail or other fixed location, a first support arm having a clamp or other fixture attachable to the handle 24 of the endoluminal access device, and a second support arm that is attachable to a handle of the endoscope 90. The first support arm and second support arm of the support stand are configured to be selectively fixed in place or to have effectively free range of motion, such as may be provided by having one or more ball joints or other pivotable connections that allow the user to selectively fix or release the system. Alternatively, the second support arm comprises a boom that is held in a fixed relationship to the first support arm, thereby allowing movement of the endoluminal access device and the endoscope 90 as a single unit. In the embodiment, a holder interface, such as a rotating clamp 300 is used to detachably attach the handle 24 to the first support arm via a post 310, thereby providing a rotational movement capability between the handle 24 and the support stand. Another holder interface, such as a C-clamp that is detachable from the second support arm, may be used to attach the endoscope 90 to the second support arm. Further, the endoluminal access device includes a plurality of instrument lumens that support slidable docking of instruments in the handle 24, with one or more of the instruments having a rigid proximal shaft section 254.

Although a number of illustrative variations are described above, it will be apparent to those skilled in the art that various changes and modifications may be made thereto without departing from the scope of the invention. Moreover, although specific configurations and applications may be shown, it is intended that the various features may be utilized in various combinations and in various types of procedures as practicable. It is intended in the appended claims to cover all such changes and modifications that fall within the true spirit and scope of the invention.

What is claimed is:

1. An endoluminal instrument management system, comprising:
an elongate body adapted to transition between a flexible state and a rigid state along its length, the elongate body having multiple lumens therethrough and a handle assembly connected to a proximal end of the elongate body, wherein the elongate body further comprises a port in communication with a lumen through the elongate body, the port being defined along the length of the elongate body distal to the handle assembly; and
a tissue manipulation assembly having a flexible shaft adapted to be advanced through the elongate body, wherein the flexible shaft of the tissue manipulation assembly comprises a rigid section near or at a proximal end of the flexible shaft, the rigid section providing structural support to a handle of the tissue manipulation assembly when positioned within the handle assembly.

2. The system of claim 1 wherein the elongate body comprises a steerable distal portion.

3. The system of claim 1 wherein the rigid section of the tissue manipulation assembly is comprised of a rigid sleeve.

4. The system of claim 1 wherein the rigid section is positioned between lengths of flexible shaft.

5. The system of claim 1 further comprising one or more visual markings positioned along the rigid section, the one or more visual markings corresponding to a depth of insertion of the tissue manipulation assembly with respect to the elongate body.

6. The system of claim 1 further comprising an imaging system having an elongate flexible shaft sized to be positioned within at least a portion of the elongate body, the imaging system having an imager disposed at its distal end.

7. The system of claim 6 wherein the imaging system comprises an endoscope.

8. The system of claim 6 wherein the imaging system is positioned through the port defined along the length of the elongate body.

9. The system of claim 1 wherein the port comprises a Y-Port.

10. The system of claim 1 further comprising a helical tissue engager having a flexible shaft which is positionable through at least one of the multiple lumens.

11. The system of claim 1 further comprising an endoscopic scissor having a flexible shaft which is positionable through at least one of the multiple lumens.

12. The system of claim 1 wherein the elongate body comprises a plurality of nested links which are adapted to compress against one another when transitioned into the rigid state.

13. An endoluminal instrument management system, comprising:
an elongate body adapted to transition between a flexible state and a rigid state along its length, the elongate body having multiple lumens therethrough and a handle assembly connected to a proximal end of the elongate body; and
a rigid channel adapted to be attached to a proximal end of the handle assembly, wherein the rigid channel defines multiple access lumens therethrough, each access lumen being in communication with a corresponding lumen through the elongate body.

14. The system of claim 13 wherein the rigid channel defines at least one straight access lumen and at least one access lumen angled or curved relative to the straight access lumen.

15. The system of claim 13 wherein the rigid channel further defines an enclosure opening within which the access lumens are positioned.

16. The system of claim 15 wherein the access lumens are pivotable within the enclosure opening relative to one another.

17. An endoluminal instrument management system, comprising:
an articulatable support arm configured to retain an elongate body which is adapted to transition between a flexible state and a rigid state along its length, the elongate body having multiple lumens therethrough and a handle assembly connected to a proximal end of the elongate body; and
at least one instrument support member configured to be articulated so as to support a corresponding instrument projecting from the handle assembly.

18. The system of claim 17 wherein the articulatable support arm is attached to a table.

19. The system of claim 17 wherein the articulatable support arm is pivotable to allow for multiple degrees-of-freedom.

20. The system of claim 17 further comprising a locking mechanism which is adapted to lock a position of the elongate body relative to the articulatable support arm.

21. The system of claim 17 wherein the at least one instrument support member is attached to the articulatable support arm.

22. The system of claim 17 wherein the at least one instrument support member is pivotable.

23. The system of claim 17 wherein the at least one instrument support member is connected to the corresponding instrument via a biased member.

24. An endoluminal instrument management system, comprising:
a curved platform having one or more attachments to a table such that the platform is positioned over at least a portion of the table; and
a plurality of instrument retaining attachments positioned over a surface of the platform,
wherein the one or more attachments are pivotable and/or translatable such that the platform is adjustable with respect to the table.
